(19) United States
(12) Patent Application Publication
(10) Pub. No.: US 2017/0212188 A1
(43) Pub. Date: Jul. 27, 2017

(54) MAGNETIC SENSOR AND MAGNETIC SENSOR APPARATUS

(71) Applicant: KABUSHIKI KAISHA TOSHIBA, Tokyo (JP)

(72) Inventors: Akira KIKITSU, Yokohama (JP); Hitoshi IWASAKI, Tokyo (JP); Satoshi SHIROTORI, Yokohama (JP)

(73) Assignee: Kabushiki Kaisha Toshiba

(21) Appl. No.: 15/258,464

(22) Filed: Sep. 7, 2016

(30) Foreign Application Priority Data
Jan. 26, 2016 (JP) 2016-012642

(51) Int. Cl. G01R 33/09; A61B 5/04; A61B 5/00; G01R 33/025

(52) U.S. Cl. CPC G01R 33/091; G01R 33/025; A61B 5/04007; A61B 5/04008; A61B 5/72

(57) ABSTRACT

A magnetic sensor of an embodiment includes: a magnetic field detector including a magnetic layer in which a length in a first direction is 10 times or more of a length in a second direction perpendicular to the first direction and a length in a third direction perpendicular to the first direction and the second direction is ½ or less of the length in the second direction; a first magnetic material member arranged along the first direction, and in which a length in the third direction is longer than the length in the third direction of the magnetic layer; a first nonmagnetic insulating layer arranged between the magnetic field detector and the first magnetic material member, and in which a length in the second direction is ½ or less of the length in the second direction of the magnetic layer; and a circuit supplying current to the magnetic layer.

### CROSS REFERENCE TO RELATED APPLICATIONS

This application is based upon and claims the benefit of priority from prior Japanese Patent Application No. 2016-012642 filed on Jan. 26, 2016 in Japan, the entire contents of which are incorporated herein by reference.

### FIELD

Embodiments described herein relate generally to a magnetic sensor and a magnetic sensor apparatus.

### BACKGROUND

Conventionally, a biomagnetic measurement apparatus using a superconducting quantum interference device (SQUID) magnetic sensor has been devised as an apparatus for measuring a magnetic field generated from a living body. The biomagnetic measurement apparatus is capable of obtaining two-dimensional biomagnetic information such as a magnetoencephalogram, or a magnetocardiogram, by arraying multiple SQUID magnetic sensors and using the sensors for biomagnetic measurement. Since the SQUID magnetic sensor uses superconductivity, it is necessary to be cooled with a refrigerant such as liquid helium. For this reason, the biomagnetic measurement apparatus using the SQUID magnetic sensor becomes huge, and operating cost is increased since the refrigerant is used, and power consumption is also increased, and restraint of a measurement subject (patient) is required.

A TMR magnetic sensor (a device using Tunnel Magneto-Resistance effect) has been known. The TMR magnetic sensor can produce a large output signal, but the noise level is large, and as a result, an SN ratio is not large.

A CIP-GMR magnetic sensor (a device using Current In Plane-Giant Magneto-Resistance effect) has been known. Since the CIP-GMR magnetic sensor has a small resistance, the noise level is small. However, the output signal is small due to the small magneto-resistance ratio. A CIP-GMR magnetic sensor with a magnetic flux concentrator (MFC) made of a soft magnetic material is provided in order to improve the output.

The CIP-GMR detects magnetic field of the direction perpendicular to its long axis. When magnetization of a field detection layer is rotated to the external field direction, a magnetic charge is generated at the side of the field detection layer and it generates a demagnetization field. The demagnetization field causes a non-linear distortion in the rotation of the magnetization, and the distortion varies with its position. This distortion causes a problem of the degradation of the SN ratio.

### BRIEF DESCRIPTION OF THE DRAWINGS

FIG. 1 is a plan view of a magnetic sensor of a first embodiment.

FIGS. 2A, 2B are diagrams schematically showing rotation of magnetization in a magneto-resistive layer due to an external magnetic field.

FIG. 3 is a diagram showing a change in a short axis direction of the magnetization of the magneto-resistive layer to an external magnetic field applied in the short axis direction.

FIG. 4 is a diagram schematically showing behavior of a magnetic field concentrator.

FIG. 5A to FIG. 5D are diagrams schematically showing the behavior of the magnetic field concentrator in the y-z plane.

FIG. 6 is a schematic diagram of a model used for a simulation to estimate a relationship between the sensitivity and a distance between the magneto-resistive layer and the magnetic field concentrator.

FIG. 7 is a diagram showing a hysteresis curve obtained by the simulation when a thickness of the magneto-resistive layer is 10 nm.

FIG. 8 is a diagram showing a hysteresis curve obtained by the simulation when a thickness of the magneto-resistive layer is 20 nm.

FIG. 9 is a diagram showing a hysteresis curve obtained by the simulation when a thickness of the magneto-resistive layer is 50 nm.

FIG. 10 is a diagram showing a simulation result of a change of μ_95 to a length of an interval gap.

FIG. 11 is a diagram showing a simulation result of a change of normalized μ_95 to the length of the interval gap.

FIG. 12 is a diagram showing a calculation result of a hysteresis loop in a case in which the length in the short axis direction of each of the magneto-resistive layer and the magnetic field concentrator is 200 nm.

FIG. 13 is a diagram showing a calculation result of a hysteresis loop in a case in which the length in the short axis direction of each of the magneto-resistive layer and the magnetic field concentrator is 200 nm. The length in a long axis direction is 2000 nm.

FIG. 14 is a plan view of a magnetic sensor of a second embodiment.

FIG. 15 is a plan view of a magnetic sensor of a modification of the second embodiment.

FIG. 16 is a plan view of a magnetic sensor of a third embodiment.

FIGS. 17A, 17B are sectional views of a magnetic field detector of the third embodiment.

FIG. 18 is a sectional view showing a first example of the magnetic field detector.

FIG. 19 is a sectional view showing a second example of the magnetic field detector.

FIG. 20 is a sectional view showing a third example of the magnetic field detector.

FIG. 21 is a sectional view showing a fourth example of the magnetic field detector.

FIG. 22 is a sectional view showing a fifth example of the magnetic field detector.

FIG. 23 is a sectional view showing a sixth example of the magnetic field detector.

FIGS. 24A to 24C are plan views for explaining a magnetic sensor production method.

FIGS. 25A to 25C are sectional views for explaining the magnetic sensor production method.

FIGS. 26A and 26B are sectional views for explaining the magnetic sensor production method.

FIG. 27A is a diagram showing a shape of a magnetic sensor used for estimation of a noise level.

FIG. 27B is a diagram showing an estimation result of the noise level.

FIG. 28 is a diagram showing a magnetic sensor apparatus of a fourth embodiment.

FIG. 29 is a diagram showing a magnetic sensor apparatus of a first modification of the fourth embodiment.

FIG. 30 is a diagram showing a magnetic sensor apparatus of a second modification of the fourth embodiment.

FIG. 31 is a diagram showing an example of a sensor unit.

### DETAILED DESCRIPTION

A magnetic sensor according to an embodiment includes: a magnetic field detector including a magnetic layer in which a length in a first direction is 10 times or more of a length in a second direction perpendicular to the first direction, and a length in a third direction perpendicular to the first direction and the second direction is ½ or less of the length in the second direction; a first magnetic material member that is arranged along the first direction of the magnetic field detector, and in which a length in the third direction is longer than the length in the third direction of the magnetic layer; a first nonmagnetic insulating layer that is arranged between the magnetic field detector and the first magnetic material member, and in which a length in the second direction is ½ or less of the length in the second direction of the magnetic layer; and a circuit configured to supply current to the magnetic layer.

Embodiments are described in detail below with reference to the drawings.

### First Embodiment

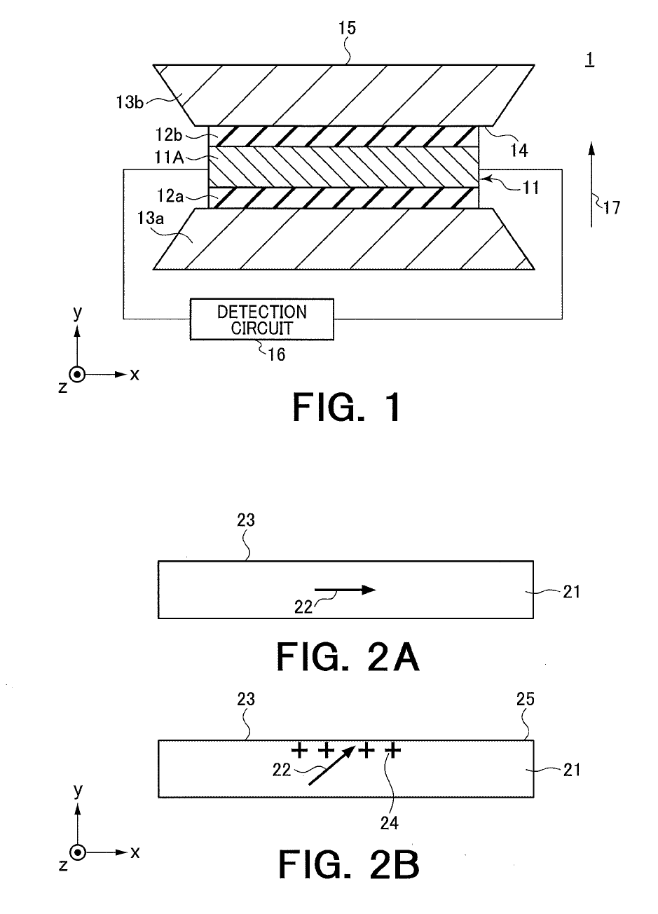

A planar schematic diagram of a magnetic sensor of a first embodiment is shown in FIG. 1. A magnetic sensor 1 of the first embodiment includes: a magnetic field detector 11; nonmagnetic insulating layers 12a, 12b provided to sandwich the magnetic field detector 11; a magnetic field concentrator 13a provided at an opposite side of the magnetic field detector 11 with respect to the nonmagnetic insulating layer 12a; and a magnetic field concentrator 13b provided at an opposite side of the magnetic field detector 11 with respect to the nonmagnetic insulating layer 12b. In the first embodiment, a magneto-resistive layer (magnetic layer) 11A is used as a magnetic field detector.

The edges of the long axis side (x direction) of the magneto-resistive layer 11A are in contact with the nonmagnetic insulators 12a, 12b respectively. Further, the nonmagnetic insulators 12a, 12b are in contact with the magnetic field concentrators 13a, 13b respectively. The magneto-resistive layer 11A is connected to a detection circuit 16 that supplies current to the magneto-resistive layer 11A in a long axis direction, and in which magnetization of the magneto-resistive layer 11A is rotated by an external magnetic field. The intensity of the magnetic field is detected by a change of electrical resistance of both ends of the magneto-resistive layer 11A. In FIG. 1, the z direction is a film thickness direction. A direction of a magnetic field to be measured is shown by an arrow 17.

The magneto-resistive layer 11A measures the magnetic field intensity utilizing a so-called magneto-resistance effect (Anisotropic Magneto-Resistive (AMR)), or, a Current In Plane-Giant Magneto-Resistance effect (CIP-GMR) phenomenon. The magnetization in the magneto-resistive layer 11A is rotated by the magnetic field to be measured, and the rotation causes a resistance change of the magneto-resistive layer 11A. It is necessary that the magnetization in the magneto-resistive layer 11A is rotated uniformly in a plane in order to obtain the best result of the phenomenon. For this reason, a ratio of a length in the long axis direction (x direction) to a length in a short axis direction (y direction) of the magneto-resistive layer 11A is preferably 10 or more.

With this configuration, the magnetization in the magneto-resistive layer 11A is uniformly oriented in the long axis direction under no external magnetic field by the shape magnetic anisotropy, unless other magnetic anisotropy is not given to the magneto-resistive layer 11A. When magnetic anisotropy is given in the short axis direction by being cooled in the magnetic field or the like, the magnetization in the magneto-resistive layer 11A is uniformly oriented in the short axis direction. When the ratio of the long axis to the short axis of the magneto-resistive layer 11A is less than 10, the shape magnetic anisotropy becomes weak and the magnetization in the magneto-resistive layer 11A is not oriented uniformly. In this case, plural magnetic domains are generated and degrade the sensitivity and the signal to the noise ratio (SNR).

A thickness (length in z direction) of the magneto-resistive layer 11A is preferably ½ or less of a length in the short axis direction (y direction). When the thickness is greater than ½ of the length in the short axis direction, the magnetization is also oriented in a direction perpendicular to a film surface, and the magnetic domain is generated. In addition, the magnetization in the magneto-resistive layer 11A does not rotate uniformly. This situation is not preferable because sensitivity is reduced and noise is increased.

The magnetic field concentrators 13a, 13b are each preferably formed of a soft magnetic material. A cross-section of the magnetic field concentrator 13a at the edge of the magneto-resistive layer 11A side (14) is less than a cross-section of its opposite side 15. Similar to the case in the magnetic field concentrator 13a, a cross-section of the magnetic field concentrator 13b at the edge of the magneto-resistive layer 11A side is less than a cross-section of its opposite side. Such different cross-sectional areas can be fabricated by making a length in the x direction of the magnetic field concentrators 13a, 13b at the edge of the magneto-resistive layer 11A side (14) less than a length in the x direction of the opposite surface 15, as shown in FIG. 1. Otherwise, such a structure can be fabricated by making a thickness (length in z direction) of the magnetic field concentrators 13a, 13b at the edge of the magneto-resistive layer 11A side (14) less than a thickness of the opposite side 15. Further, these two techniques may be combined.

The magnetic field concentrator may be provided to one side of the magneto-resistive layer 11A. In this case, a production process is simplified, but a magnetic field concentration effect is decreased.

The magnetic field concentrators 13a, 13b used in the first embodiment are used as generally called MFC. That is, magnetic flux lines arrived at the edge 15 of the magnetic sensor 1 are concentrated to the opposite side surface 14 in the magnetic field concentrator 13a. Thus, magnetic flux density, that is, the magnetic field intensity is increased with a ratio of the cross-sections. The magnetic field concentrators 13a, 13b improve the sensitivity of the magnetic sensor 1 of the present embodiment.

Nonmagnetic insulators 12a, 12b are provided respectively between the magnetic field concentrators 13a, 13b and the magneto-resistive layer 11A.

When the nonmagnetic insulators 12a, 12b are not provided, the magnetic field concentrators 13a, 13b and the magneto-resistive layer 11A are electrically connected to each other, and electric current flowing through the magneto-resistive layer 11A may diffuse into the magnetic field concentrators 13a, 13b. This situation decreases SNR. In addition, when the nonmagnetic insulators 12a, 12b are not provided, the magneto-resistive layer 11A and the magnetic field concentrators 13a, 13b are exchange-coupled, and the rotation of the magnetization of the magneto-resistive layer 11A distorted. This situation decreases SNR. Therefore, each of the nonmagnetic insulators 12a, 12b is preferably a nonmagnetic insulator.

A length in the long axis direction (x direction) of each of the nonmagnetic insulators 12a, 12b may be the same as the length of the magneto-resistive layer 11A, and may be greater than that. A length in the short axis direction (y direction) is preferably 50 nm or less, or ½ or less of the length in the short axis direction of the magneto-resistive layer 11A. It is not preferable that the length in the short axis direction (y direction) of each of the nonmagnetic insulators 12a, 12b is 1 nm or less because they are difficult to form continuous thin film insulators. A thickness (length in z direction) of each of the nonmagnetic insulators 12a, 12b may be the same as the thickness of the magneto-resistive layer 11A, and may be greater than that.

Other magnetic layers or other nonmagnetic layers may be provided between the magnetic field concentrators 13a, 13b and the nonmagnetic insulators 12a, 12b, or between the nonmagnetic insulators 12a, 12b and the magneto-resistive layer 11A, unless they do not degrade the magnetic flux concentration effect of the magnetic field concentrators 13a, 13b and the rotation of the magnetization of the magneto-resistive layer 11A.

The length (length in x direction) of the surface in the magneto-resistive layer 11A side of the magnetic field concentrators 13a, 13b may be longer than the length (length in x direction) of the magneto-resistive layer 11A, or may be shorter than that. When the length of each of the magnetic field concentrators 13a, 13b is longer, magnetic flux is partially lost and the magnetic field intensity is decreased. However, the disturbance of the magnetic field at the edge of the magnetic field concentrators 13a, 13b can be suppressed.

When the length in the x direction of each of the magnetic field concentrators 13a, 13b is shorter than the length in the x direction of the magneto-resistive layer 11A, all the concentrated magnetic flux can be detected. However, non-uniform magnetic field in the magnetic sensor 1 may impair linearity of the magnetic sensor 1. The length of the surface 14 in the magneto-resistive layer 11A side of each of the magnetic field concentrators 13a, 13b should be determined based on requirement specification of the magnetic sensor 1 to be created.

A length in the y direction of each of the magnetic field concentrators 13a, 13b is not particularly limited; however, the length is preferably longer than a length in the y direction of the magneto-resistive layer 11A since the magnetic field concentration effect is expected to be improved.

A function of the present embodiment shown in FIG. 1 is described below in which low noise and high sensitivity magnetic field detection is achieved. FIGS. 2A, 2B are diagrams schematically showing the rotation of magnetization 22 in a magneto-resistive layer 21 by the external magnetic field. FIG. 2A shows a case of no external magnetic field. In this case, the magnetization 22 is uniformly oriented in a longitudinal direction, and a magnetic charge does not appear at the edge 23 of the long axis side of the magneto-resistive layer 21.

FIG. 2B shows a case in which the external magnetic field is applied in the short axis direction. In this case, the magnetization 22 is rotated as shown in FIG. 2B, and a magnetic charge 24 is generated at the edge of the long axis side 23 of the magneto-resistive layer 21. The magnetic charge 24 causes a demagnetization field, that is, a leakage magnetic field from the magnetic charge 24. The demagnetization field changes the orientation of the magnetization 22. Since the demagnetization field caused by the magnetic charge 24 is localized, it creates magnetic domains in the magneto-resistive layer 21, and the magnetization rotation is distorted. The distortion of the magnetization rotation is significant especially at around an end portion 25 of the magneto-resistive layer 21.

When such a phenomenon occurs, the rotation of the magnetization becomes nonlinear with respect to the external magnetic field, and accurate magnetic field measurement cannot be achieved. This is schematically shown in FIG. 3. FIG. 3 shows a change of magnetization M along the short axis direction of the magneto-resistive layer 21 with an intensity of the external magnetic field H. In a case of an ideal magnetic sensor, the magnetization M is linearly increased with the magnetic field, and is saturated at the saturation magnetic field Hk, as shown by a graph 31. Since the magnetic field intensity H and the magnetization M have an accurate proportional relationship at the magnetic field less than Hk, accurate magnetic field measurement can be achieved.

In a case in which the magnetization rotation becomes nonlinear by the magnetic charge as shown in FIG. 2B, the characteristic becomes the one shown by a graph 32, for example. Since the magnetization M is nonlinearly changed with the magnetic field H, accuracy of magnetic field measurement is degraded. In addition, the magnetization M at the same magnetic field H differs between the case of increasing the magnetic field and the case of decreasing the magnet field, that is, hysteresis occurs. This situation degrades repeatability and reliability of the measurement.

In general, in order to prevent such a nonlinear phenomenon by the magnetic charge, high magnetic anisotropy in the long axis direction is given. In this case, the demagnetization field from the magnetic charge is relatively decreased. However, the saturation magnetic field Hk is increased as shown by a graph 33 (in FIG. 3, the Hk is out of the range of the graph), and the change of the magnetization M to the magnetic field H is decreased. As a result, magnetic field sensitivity is degraded.

As described above, in the case in which magnetic field measurement is performed by the magnetization rotation in the magneto-resistive layer 11A, there has been a problem that measurement accuracy is degraded due to the magnetic charge caused at the edge of the long axis side.

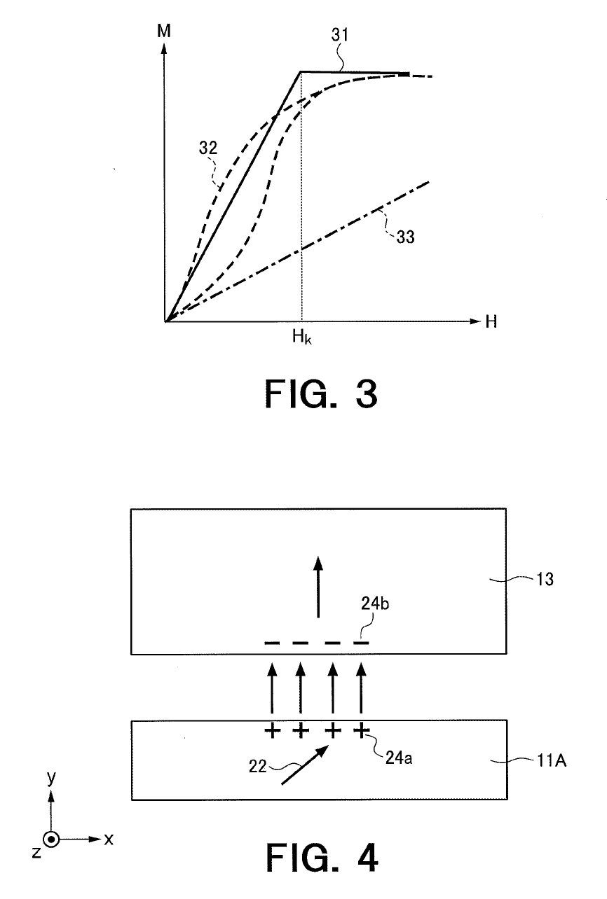

In the present embodiment, the problem is solved by providing the magnetic field concentrators 13a, 13b. That is, the magnetic field concentrators 13a, 13b are respectively provided at the edge along the long axis of the magneto-resistive layer 11A via the nonmagnetic insulators 12a, 12b. A schematic diagram for explaining this portion is shown in FIG. 4. The magnetization of the magneto-resistive layer 11A is rotated by the external magnetic field, and a magnetic charge 24a is caused. The magnetic charge 24a is shown by a symbol “+.” A magnetic field concentrator 13 made of a soft magnetic material is provided in close proximity to the magneto-resistive layer 11A.

Since the magnetic field concentrator 13 is a soft magnetic material, the magnetization at the edge of the magneto-resistive layer 11A side of the magnetic field concentrator 13 is rotated by the leakage magnetic field from the magnetic charge 24a of the magneto-resistive layer 11A, and an opposite direction magnetic charge 24b is caused. This magnetic charge 24b is shown by a symbol “−.” Since the magnetic flux lines connect the magnetic charges 24a and 24b directly, the leakage magnetic field (demagnetizing field) that distorts the magnetization rotation of the magneto-resistive layer 11A disappears.

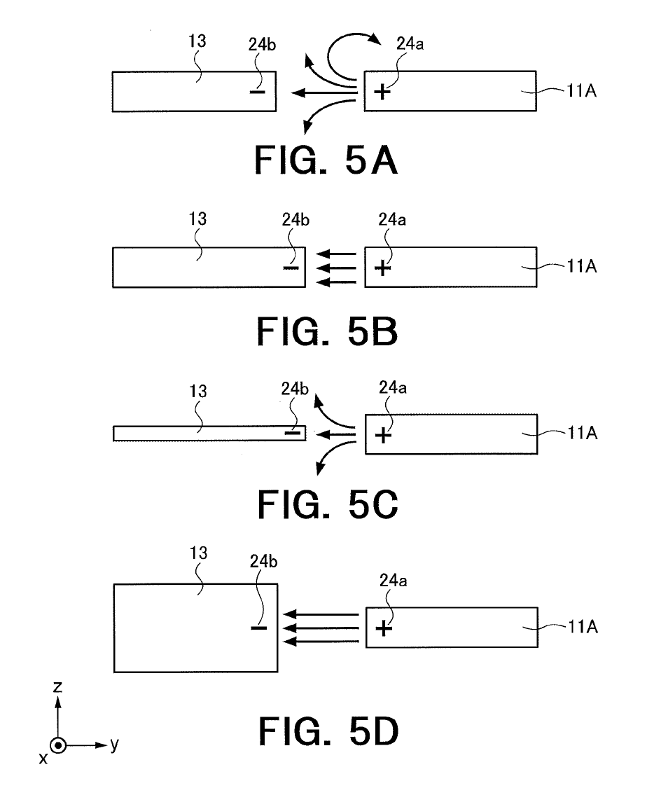

FIG. 5A to FIG. 5D show the magneto-resistive layer 11A and the magnetic field concentrator 13 in the y-z plane. FIG. 5A shows a case in which the magneto-resistive layer 11A and the magnetic field concentrator 13 have the same thicknesses and are separated by a distance. Not all the leakage magnetic field from the magnetic charge 24a is absorbed by the magnetic charge 24b of the magnetic field concentrator 13, and the leakage magnetic field partially leaks to the outside.

When the distance between the magneto-resistive layer 11A and the magnetic field concentrator 13 is sufficiently small, as shown in FIG. 5B, the magnetic flux that leaks to the outside is reduced, and the demagnetization field can be cancelled.

When the thickness (length in z direction) of the magnetic field concentrator 13 is thinner than the thickness (length in z direction) of the magneto-resistive layer 11A as shown in FIG. 5C, the demagnetization field leaks around the magneto-resistive layer 11A even when the distance between the magneto-resistive layer 11A and the magnetic field concentrator 13 is sufficiently small.

On the contrary, when the thickness (length in z direction) of the magnetic field concentrator 13 is thicker than the thickness (length in z direction) of the magneto-resistive layer 11A as shown in FIG. 5D, the demagnetization field can be more stably cancelled.

(Relationship Between Magnetic Field Sensitivity and Distance Between Magneto-Resistive Layer and Magnetic Field Concentrator)

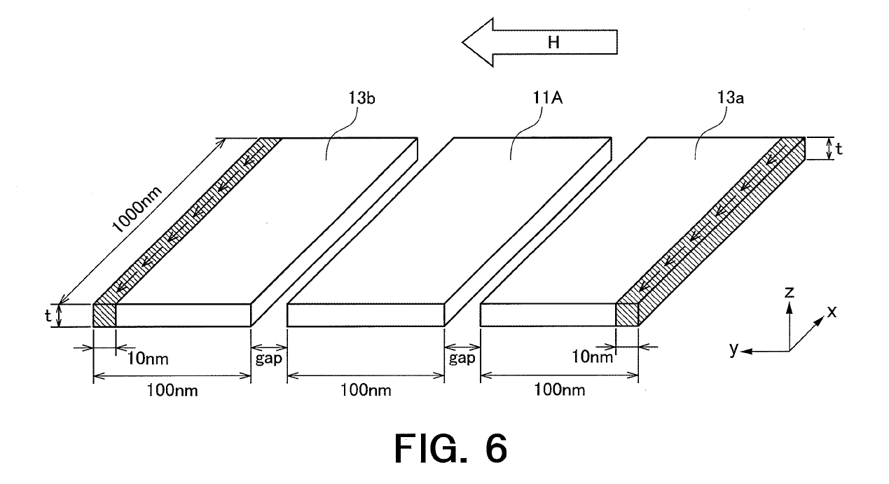

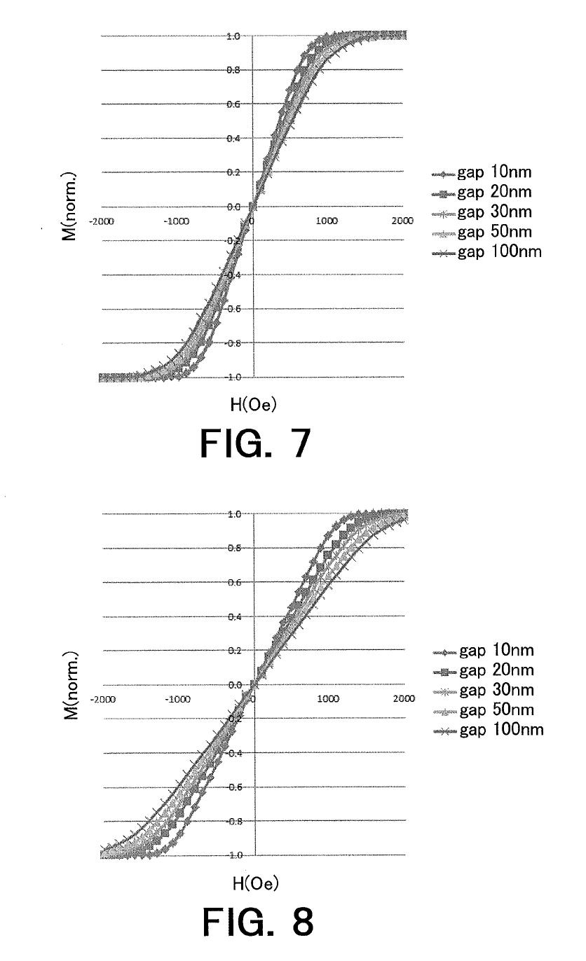

Next, a relationship between magnetic field sensitivity and a distance between the magneto-resistive layer 11A and each of the magnetic field concentrators 13a, 13b is investigated by a simulation using LLG (Landau-Lifshitz-Gilbert) equation. A schematic diagram of a model used for the simulation is shown in FIG. 6. As the magneto-resistive layer 11A, a model in which its width is 100 nm, length is 1000 nm (ratio of the long axis to the short axis is 10), and thickness t is changed to be 10, 20, and 50 nm is used. The model is divided by a 10 nm×10 nm×10 nm cube mesh. Material properties of NiFe are used for the magneto-resistive layer 11A. Saturation magnetization Ms is 800 emu/cc and magnetic anisotropy Ku_{y} in the y direction is 1 kerg/cc. An exchange constant A between the meshes is 1 μerg/cm, and a Gilbert damping constant α is 1.0. Models of the magnetic field concentrators 13a, 13b are the same size and the same magnetic characteristic as those of the magneto-resistive layer 11A, and are placed at a predetermined interval (gap) on the same plane as shown in FIG. 6. Five types of intervals gap, 10, 20, 30, 50, 100 nm, have been prepared.

It has been difficult to model the magnetic field concentrators 13a, 13b with a large size close to the size of the present embodiment since enormous calculation time is required. Then, magnetization at the end portions of 10 nm (1 mesh) of an opposite side to the magneto-resistive layer 11A of each of the magnetic field concentrators 13a, 13b is pinned in the x direction with the magnetic field of 20 kOe. With this operation, the leakage magnetic field from the magnetic charge at the end portion of the magnetic field concentrators 13a, 13b is suppressed. This situation makes the magnetic field concentrators 13a, 13b similar to a soft magnetic material infinitely long in the y direction. This operation is generally used for a soft magnetic material.

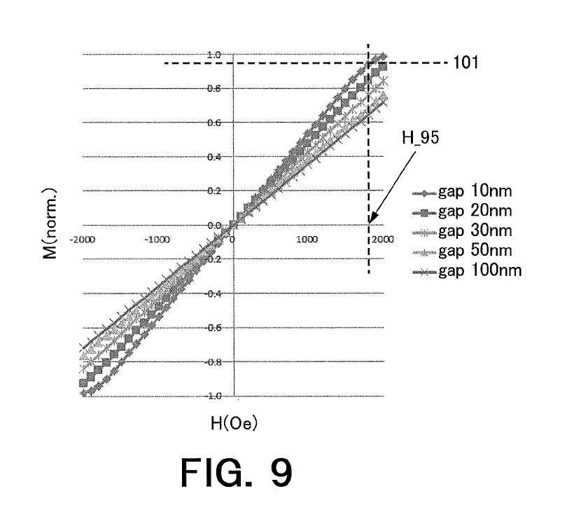

Calculated hysteresis loops are shown in FIG. 7 to FIG. 9. The horizontal axis indicates applied magnetic field and the vertical axis indicates normalized magnetization in the y direction of the magneto-resistive layer 11A. The external magnetic field has been applied in the y direction as shown in FIG. 6. Y direction components of the magnetization of a center portion (rectangular portion of 500 nm×100 nm in the x-y plane) of the magneto-resistive layer 11A are plotted. Thus, the obtained hysteresis loop corresponds to GMR or AMR output in which an artifact noise due to a small calculation area is excluded. FIG. 7 shows a hysteresis loop of the case of the thickness t is 10 nm. FIG. 8 and FIG. 9 show hysteresis loops for the case of t=20 nm and t=50 nm, respectively. In all cases, relatively excellent linearity with little hysteresis is shown. Linearity and hysteresis are improved as the film thickness t reduced.

This means almost no magnetization rotation in the film thickness direction of the magneto-resistive layer 11A. Thus, it has been shown that an excellent soft magnetic characteristic is obtained when the film thickness of the magneto-resistive layer 11A is ½ or less of the length (=100 nm) in the short axis direction.

As an index of the magnetic field sensitivity, a magnetic field H_95, where the magnetization reaches 95% of the saturation magnetization Ms, and its reciprocal 1/(H_95)=μ_95 are defined. A procedure for obtaining the H_95 is schematically shown in FIG. 9. A line 101 indicates the magnetization with 95% of its saturation value, and a magnetic field at the position is the H_95. Magnetic field sensitivity is high when H_95 is small, that is, when μ_95 is large.

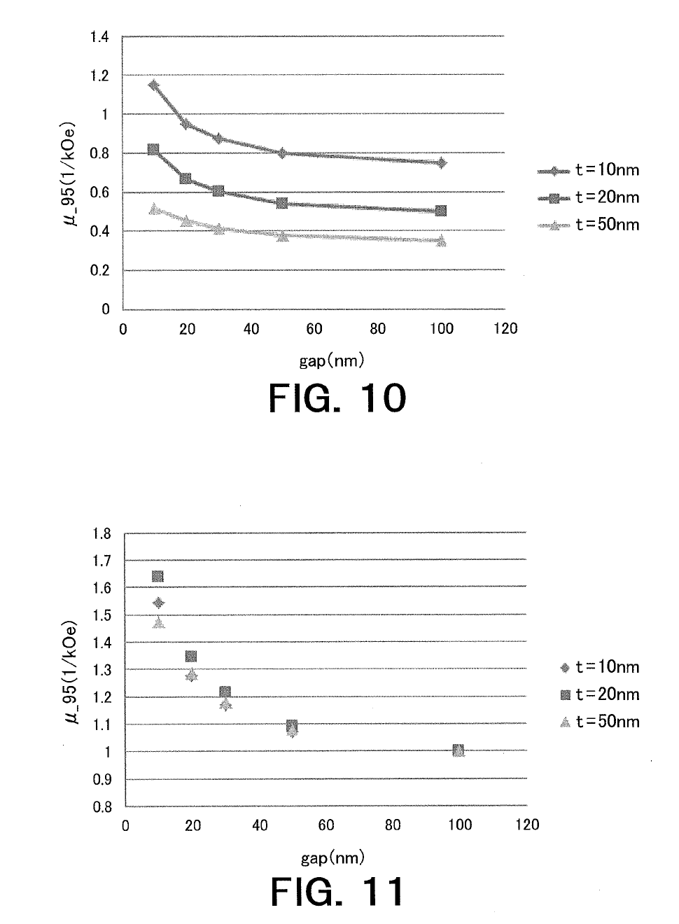

A change of the μ_95 with the gap is shown in FIG. 10. FIG. 11 shows a graph normalized by a value of when the gap is 100 nm. As can be seen from FIG. 11, in any film thickness, the μ_95 is drastically increased for the gap less than 50 nm. In particular, when the gap is less than 20 nm, the μ_95 is increased by 20% or more. This is because, as described in FIGS. 5A to 5D, the distance between each of the magnetic field concentrators 13a, 13b and the magneto-resistive layer 11A is decreased, and the demagnetization field has been suppressed. From results of FIGS. 10, 11, it has been found that this effect significantly appears in a case in which the distance between the magneto-resistive layer 11A and each of the magnetic field concentrators 13a, 13b is 50 nm or less, more preferably 20 nm or less, that is, in a case in which the distance is ½ or less of the width of the magneto-resistive layer 11A. In a case in which the distance between the magneto-resistive layer 11A and each of the magnetic field concentrators 13a, 13b is 0 nm, that is, there are no nonmagnetic insulators 12a, 12b, it is not preferable because the SN ratio of the output signal is degraded due to the electric current diffusion and non-uniform magnetization rotation, as described above.

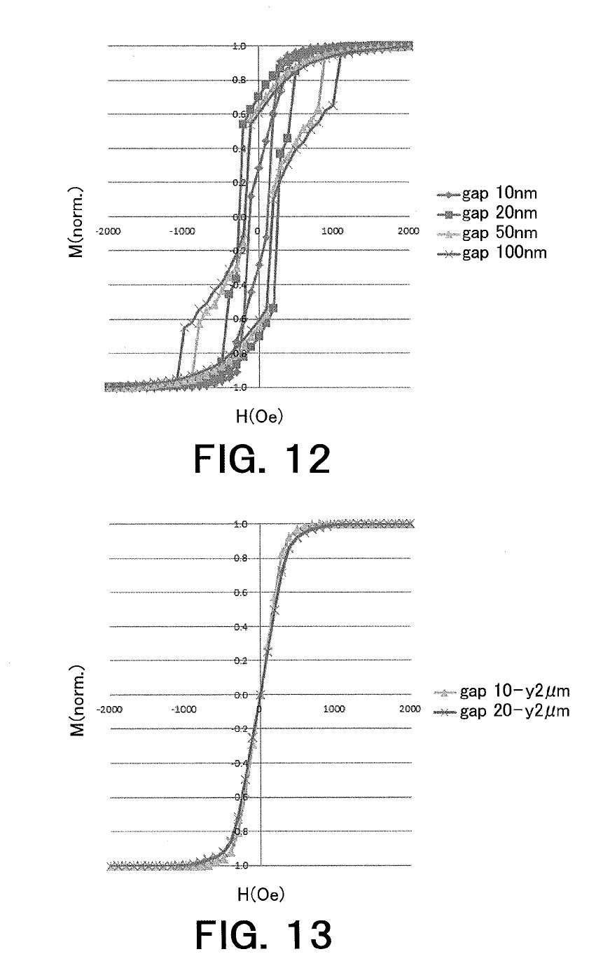

Next, calculation has been performed in a case in which the length (width) in the short axis direction (y direction) of the magneto-resistive layer 11A and each of the magnetic field concentrators 13a, 13b is 200 nm. A result is shown in FIG. 12. Film thickness t is 10 nm.

A calculation area of the hysteresis loop is a center portion of the magneto-resistive layer 11A, that is, the rectangular portion of 500 nm×200 nm in the x-y plane. As can be seen from FIG. 12, nonlinearity, that is, a large hysteresis has appeared. This means that, the magnetization in the magneto-resistive layer 11A is not rotated uniformly since the ratio of the long axis to the short axis of the magneto-resistive layer 11A is less than 10, as described above. This tendency is also observed for the cases when the film thickness t is 20, 50, or 100 nm.

Next, a result is shown in FIG. 13 for a case in which the length (width) in the short axis direction (y direction) of the magneto-resistive layer 11A and each of the magnetic field concentrators 13a, 13b is 200 nm, the thickness t is 10 nm, and the length in the long axis direction (x direction) of the magneto-resistive layer 11A and each of the magnetic field concentrators 13a, 13b is 2000 nm. A calculation area of the hysteresis loop is a center portion of the magneto-resistive layer 11A, that is, the rectangular portion of 1000 nm×200 nm in the x-y plane. Due to the long calculation time, only the cases of gap=10 nm and 20 nm are simulated. Nonlinearity shown in FIG. 12 has disappeared. It has been found that the ratio of the long axis to the short axis of the magneto-resistive layer 11A is preferably 10 or more.

As described above, with the first embodiment, nonlinearity can be suppressed, and a magnetic sensor with less noise and high sensitivity can be achieved.

### Second Embodiment

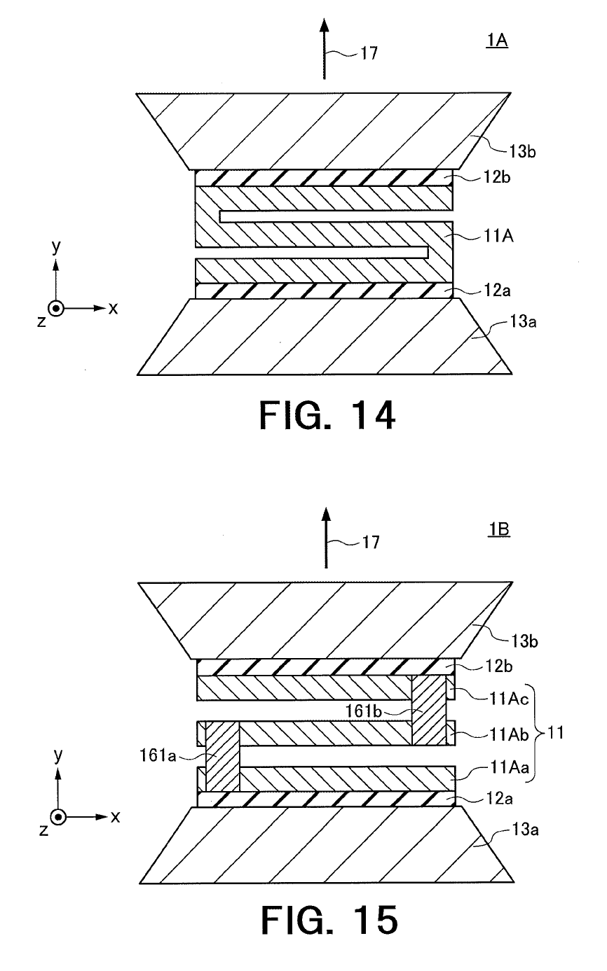

Next, a magnetic sensor of a second embodiment is described with reference to FIG. 14. FIG. 14 is a plan view of a magnetic sensor 1A of the second embodiment.

Generally, in a current in plane type magnetic sensor, the magneto-resistive layer 11A should be long in order to improve its SN ratio. However, simply long sensor is not suitable for a small form factor sensor. A magnetic sensor in which this problem has been solved is described as the second embodiment.

The magnetic sensor 1A of the second embodiment has a configuration in which, a magneto-resistive layer 11A is folded and made into a zigzag shape as shown in FIG. 14. In FIG. 14, the magneto-resistive layer is folded twice, and a length in the x direction of the entire magnetic sensor 1A can be reduced to ⅓ in comparison with the length in a case in which the magneto-resistive layer is not folded.

Also in the second embodiment, similarly to the first embodiment, a magnetic sensor with low noise and high sensitivity can be obtained as follows. As shown in FIG. 14, at the outermost portions of the magneto-resistive layer 11A, nonmagnetic insulators 12a, 12b and magnetic field concentrators 13a, 13b each made of a soft magnetic material are respectively placed in this order along the long axis side. The width of each of the nonmagnetic insulators 12a, 12b, that is, a distance between the magneto-resistive layer 11A and each of the magnetic field concentrators 13a, 13b is 50 nm or less (½ or less of a length in a short axis direction of the magneto-resistive layer 11A). Requirements that should be satisfied by the magnetic field concentrators 13a, 13b are the same as those of the first embodiment.

A function to achieve low noise and high sensitivity magnetic field detection is the same as that of the first embodiment. The magnetic field concentrators 13a, 13b each made of the soft magnetic material each have a function that cancels a demagnetization field by a magnetic charge generated at the edge of the magneto-resistive layer 11A. Thus, the nonlinear rotation of magnetization is prevented.

Therefore, the second embodiment, similarly to the first embodiment, also provides a magnetic sensor with low noise and high sensitivity.

(Modification)

A magnetic sensor of a modification of the second embodiment is shown in FIG. 15. A magnetic sensor 1B of this first modification has a configuration in which the zigzag-shaped magneto-resistive layer 11A of the second embodiment shown in FIG. 14 is replaced by three magneto-resistive layers 11Aa, 11Ab, 11Ac separately placed in parallel, and adjacent two magneto-resistive layers 11Aa, 11Ab are connected to each other in series with a conductor 161a, and adjacent two magneto-resistive layers 11Ab, 11Ac are connected to each other in series with a conductor 161b. This configuration is electrically the same as that of the magneto-resistive layer 11A of the second embodiment shown in FIG. 14.

In a case of the magneto-resistive layer HA with the zigzag shape shown in FIG. 14, magnetization is not rotated by the magnetic field at a portion where the direction of the magneto-resistive layer is parallel to the direction of a magnetic field 17. Therefore, contribution of this portion to the sensitivity is small. For this reason, the function of a magnetic sensor without this portion is almost the same as that of the second embodiment. Rather, since the nonlinear magnetization rotation can be suppressed, this configuration can provide lower noise sensing properties.

The modified configuration shown in FIG. 15 also provides a magnetic sensor capable of low noise and high sensitivity magnetic field detection as in the cases of the second embodiment. That is, the nonmagnetic insulators 12a, 12b and the magnetic field concentrators 13a, 13b each made of the soft magnetic material are respectively placed in this order to the long axis side end of the outermost magneto-resistive layers 11A. The width of each of the nonmagnetic insulators 12a, 12b, that is, a distance between the magneto-resistive layer 11A and each of the magnetic field concentrators 13a, 13b is set to be 50 nm or less (½ or less of the length in the short axis direction of the magneto-resistive layer 11A). Requirements that should be satisfied by the magnetic field concentrators 13a, 13b are the same as those of the first embodiment. A function in which the magnetic sensor is capable of low noise and high sensitivity magnetic field detection is the same as that of the first embodiment.

In addition, a mixed form of the magneto-resistive layers 11A shown in FIG. 14 and FIG. 15 may be possible. For example, the conductors 161a, 161b may be placed on the zigzag-shaped magneto-resistive layer. In that case, the magneto-resistive layers 11A under the conductors 161a, 161b may partially connected to each other.

This additionally modified embodiment is also capable of providing a magnetic sensor that suppresses noise and has a high sensitivity.

### Third Embodiment

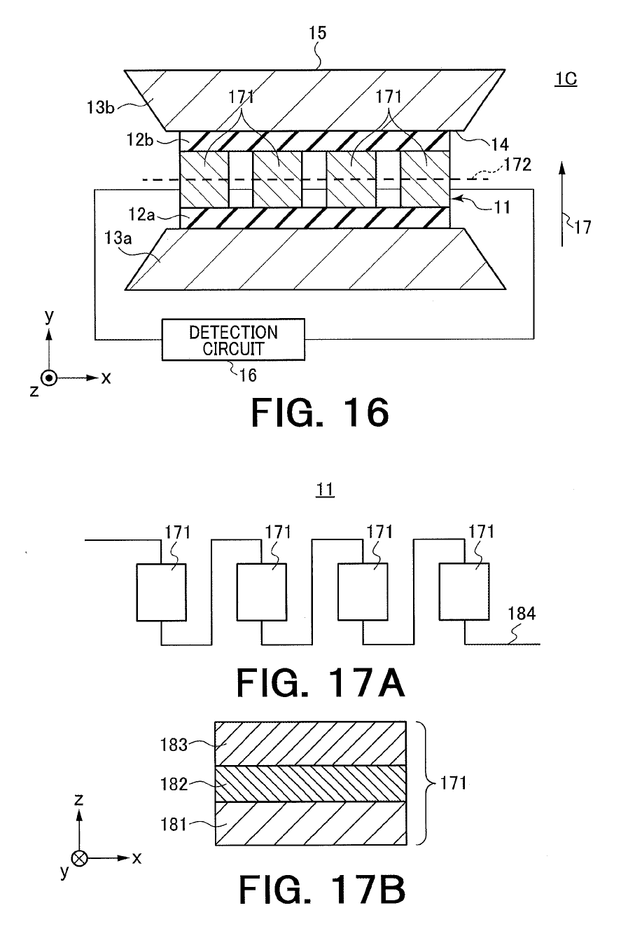

A plan view of a magnetic sensor of a third embodiment is shown in FIG. 16. A magnetic sensor 1C of the third embodiment is configured so that, in the magnetic sensor 1 of the first embodiment shown in FIG. 1, a magnetic field detector 11 is replaced with current perpendicular to plane GMR devices 171 connected to each other in series in the in-plane direction. An electric current flows in the in-plane direction through an entire device. One current perpendicular to plane GMR device 171 may be used as the magnetic field detector 11 though the sensitivity of the magnetic field is degraded.

The y direction end sides of the current perpendicular to plane GMR devices 171 are in contact with nonmagnetic insulators 12a, 12b. FIG. 17A shows an x-z cross section at a position of a dotted line 172 shown in FIG. 16. The current perpendicular to plane GMR devices 171 are connected to each other in series by the conducting wire 184. A detailed configuration of the x-z plane of each of the current perpendicular to plane GMR devices 171 is shown in FIG. 17B. Each of the current perpendicular to plane GMR devices 171 has a configuration in which a pinned layer 181 made of a magnetic material, a nonmagnetic interlayer 182, and a free layer 183 made of a magnetic material are layered in this order.

Each of the pinned layer 181 and the free layer 183 is a magnetic material in which a magnetization direction is parallel to a film surface, that is, a magnetic material in which magnetization is rotated in the x-y plane. The pinned layer 181 is designed so that the magnetization is not easily rotated by an external magnetic field. For this purpose, an antiferromagnetic material (not shown) with a high magnetic anisotropy is provided under the pinned layer 181, where the magnetization direction of the pinned layer 181 is aligned to that of the antiferromagnetic material by exchange coupling.

The free layer 183 is a magnetic material having a characteristic in which the magnetization is easily rotated by a measured magnetic field. For this purpose, a material having soft magnetic properties is used. The nonmagnetic interlayer 182 is selected from metals or insulators. Thin insulator such as MgO may be used as nonmagnetic interlayer 182 (generally referred to as TMR), I It is preferable to use the TMR device since TMR has a large magneto-resistance ratio.

A function in which the magnetic sensor 1C provides low noise and high sensitivity properties is the same as that of the first embodiment. Responses of the magnetization to the external magnetic field are the same between a case of the magneto-resistive layer 11A and a case of the free layer 183. When the magnetization in the free layer 183 is rotated by the external magnetic field, each of the magnetic field concentrators 13a, 13b made of a soft magnetic material has a function that cancels a demagnetization field from a magnetic charge generated at the end portion of the free layer 183. Thus, nonlinear rotation of the magnetization in the free layer 183 is suppressed.

By making a width (a distance between the GMR devices 171 and each of the magnetic field concentrators 13a, 13b) of each of the nonmagnetic insulators 12a, 12b be 50 nm or less, or ½ or less of a length in a short axis direction of the free layer 183, a demagnetization field can be cancelled effectively.

In addition, when in-plane arrangement of the current perpendicular to plane GMR devices 171 is a zigzag shape and the devices are connected to each other in series, the same function as of the second embodiment can be obtained.

As a material of the free layer 183, a FeCo alloy or a Heusler alloy is preferable, in which high spin polarization is obtained and a magneto-resistance ratio is increased. The pinned layer 181 consists of enhancing material with high magnetic crystalline anisotropy. Example is an alloy such as CoPt, CoPd, or an ordered phase alloy such as FePt, CoPt, CoPd, or a so-called super lattice structure such as Co/Pd, Co/Pt, Co/Ni in which multiple ultrathin magnetic layers are stacked. In addition, a soft magnetic material may be used for the pinned layer 181. In this case, an antiferromagnetic layer or a high magnetic anisotropy layer is placed under the pinned layer 181 to exchange-coupled with the pinned layer 181.

Nonmagnetic metal containing Cu may be used as the nonmagnetic interlayer 182. For the case in which the TMR device is used, Al—O, Ti—O, MgO, or the like may be used as the nonmagnetic interlayer 182.

(Material of Constituents)

Next, a material of each component configuring the first to third embodiments is described.

The magneto-resistive layer 11A is required to have an excellent soft magnetic characteristic, that is, a characteristic in which the magnetization is rotated even by a small magnetic field, and also to have a magnetic characteristic that shows a large magneto-resistance ratio. Detailed constituent features of the magnetic field detector are described later.

Any nonmagnetic insulator of insulators used for general devices may be used for the nonmagnetic insulators 12a, 12b. In particular, a compound of metal and gas such as oxygen or nitrogen is generally used, such as Si—O, Si—N, Al—O, Mg—O. Although an oxide of Fe has magnetic properties depending on its composition, the oxide of Fe can be used if it is nonmagnetic. An oxide of the materials used for the magneto-resistive layer 11A, the magnetic field concentrators 13a, 13b, or a protective layer (described later) may be used for the nonmagnetic insulators 12a, 12b.

Each of the magnetic field concentrators 13a, 13b is preferably a magnetic material having an excellent soft magnetic characteristic similar to the magneto-resistive layer 11A. For example, a material containing a NiFe alloy, a FeCo alloy, or a Heusler alloy may be used.

As a detection circuit 16 that detects a resistance change, it is possible to use a circuit used for a general magneto-resistive sensor device. An example of the circuit has a system in which, electric current is applied to the magneto-resistive layer 11A from a DC current source along its long axis direction and a voltage across the both ends of the magneto-resistive layer 11A is measured. Since each of the magnetic sensors of the first to third embodiments is highly sensitive, it is necessary to use a low noise current source and a low noise amplifier.

For each of the magneto-resistive layer 11A, the nonmagnetic insulators 12a, 12b, and the magnetic field concentrators 13a, 13b, a base layer may be used for improving crystallinity and/or magnetic characteristics. In addition, for the purpose of preventing process damage such as resist coating, a protective layer may be deposited successively after film deposition of the magnetic layers describe above. The protective layer may be removed during the process or may not be removed, unless the protective layer does not affect functions of the magnetic sensor.

(Magnetic Field Detector)

Next, an example of the magnetic field detector 11 is described.

### First Example

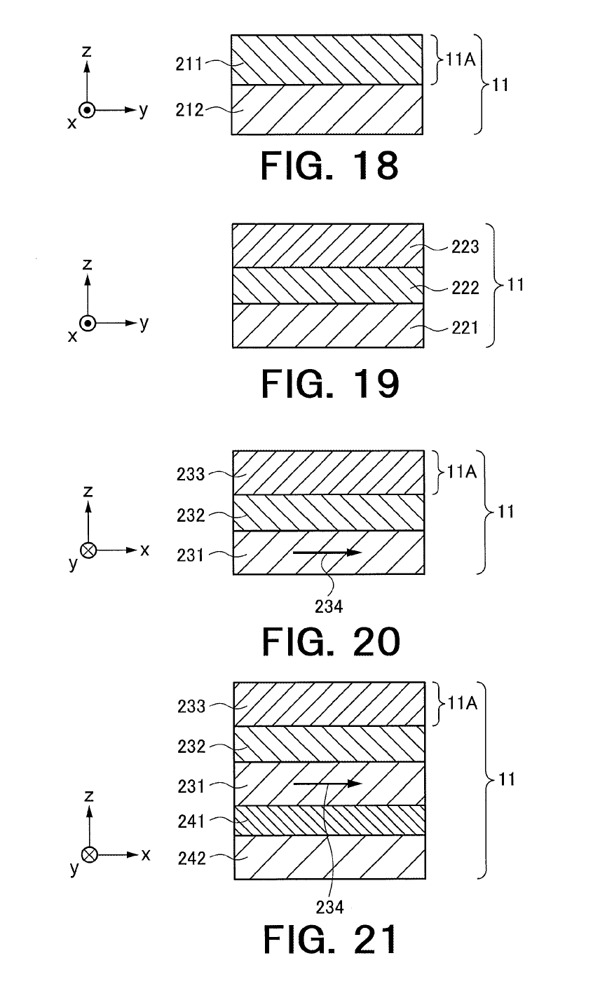

The first example of the magnetic field detector 11 for the first to second embodiments consists of a soft magnetic material 211 that includes any of a NiFe alloy, a FeCo alloy, and a Heusler alloy. And the soft magnetic material 211 may be formed on a nonmagnetic base layer 212 such as NiFeCr, as shown in FIG. 18.

As shown in FIG. 18, the soft magnetic material 211 corresponds to the magneto-resistive layer 11A of the first to second embodiments. In this case, the resistance change is caused by so-called Anisotropic Magneto-Resistive (AMR).

Since an AMR device has a simple structure, a low cost and high reliability magnetic sensor can be produced. In addition, the AMR device is a low noise device since its electrical resistance is low. However, since the magneto-resistance ratio is small, the sensitivity is not very high.

### Second Example

The second example of the magnetic field detector 11 for the first to second embodiments consists of a first magnetic layer 221 made of a soft magnetic material, a nonmagnetic conductive layer 222, and a second magnetic layer 223 made of a soft magnetic material stacked in this order, as shown in FIG. 19. The first magnetic layer 221 and the second magnetic layer 223 are antiferromagnetically coupled together via the nonmagnetic conductive layer 222. As indicated in FIG. 19, a stacked portion of the first magnetic layer 221, the nonmagnetic conductive layer 222, and the second magnetic layer 223 corresponds to the magneto-resistive layer 11A.

The second example of the magnetic field detector 11 is referred to as a scissors-type CIP-GMR device. Since the first magnetic layer 221 and the second magnetic layer 223 are antiferromagnetically exchange-coupled together, magnetization of each layer is oriented in opposite directions to each other in the long axis direction when there is no external magnetic field. When a magnetic field is applied in a short axis direction of the magnetic field detector 11, the magnetization of each layer is rotated to the short axis direction, and an angle between the magnetizations of both layers 221, 223 is decreased from 180 degrees. This angle change is detected as a resistance change.

As the nonmagnetic conductive layer 222, a nonmagnetic metal containing any of Cu, Ru, or Ir may be used. In order to induce antiferromagnetic coupling between the first magnetic layer 221 and the second magnetic layer 223, the thickness of the nonmagnetic conductive layer 222 should be about 2 nm or less.

For the first magnetic layer 221 and the second magnetic layer 223, magnetic anisotropy may be given in the short axis direction by such as annealing in the magnetic field. This process makes the magnetizations of the first and second magnetic layers 221, 223 have an angle smaller than 180 degrees under zero external magnetic field. When the external magnetic field is applied, the angle becomes further smaller.

This example is preferable since the CIP-GMR device shows the maximum magneto-resistance ratio when the angle between the magnetizations of the layers is around 90 degrees. However, a fabrication cost may be increased by an additional process for giving the anisotropy in the short axis direction.

For the first magnetic layer 221 and the second magnetic layer 223, a FeCo alloy or a Heusler alloy is preferable since they have high spin polarization and show high magneto-resistance ratio.

### Third Example

The third example of the magnetic field detector 11 for the first to second embodiments consists of a pinned layer 231 in which the magnetization is pinned in the long axis direction, a free layer 233 made of a soft magnetic material in which the magnetization is rotated by the external magnetic field, and a nonmagnetic conductive layer 232 provided between the pinned layer and the free layer, as shown in FIG. 20. As indicated in FIG. 20, the free layer 233 corresponds to the magneto-resistive layer 11A of the first to second embodiments. In FIG. 20, an arrow 234 indicates that the magnetization is pinned in the long axis direction.

Such a configuration is referred to as a spin-valve type CIP-GMR device. The magnetization of the free layer 233 is rotated by the external magnetic field, and its angle to a direction of the magnetization of the pinned layer 231 is changed. This angle change is detected as a resistance change. As the nonmagnetic conductive layer 232, a nonmagnetic metal containing Cu may be used.

For pinning the magnetization of the pinned layer 231, magnetocrystalline anisotropy or shape magnetic anisotropy is generally used.

The magnetic sensor for the third example of the magnetic field detector 11 has a long axis, so that a large shape magnetic anisotropy is generated in the long axis direction, shown by the arrow 234 in FIG. 20.

Anisotropy may be given in the short axis direction of the free layer 233 by the process such as annealing in the magnetic field. This configuration is preferable because an angle between the magnetization of the free layer 233 and the magnetization of the pinned layer 231 becomes around 90 degrees under zero magnetic field, as described above. However, fabrication cost may be increased.

As the free layer 233 and the pinned layer 231, a FeCo alloy or a Heusler alloy is preferable since they have high spin polarization and provide high magneto-resistance ratio. As a material with high magnetocrystalline anisotropy used for the pinned layer 231, an alloy such as CoPt, CoPd, or an ordered phase alloy (such as FePt, CoPt, CoPd), or a so-called super lattice film (such as Co/Pd, Co/Pt, Co/Ni in which multiple ultrathin magnetic layers are layered) may be used.

Next, a method to fix the magnetization of the pinned layer 231, which is used for the third example of the magnetic field detector 11 shown in FIG. 20, is described.

In order to orient the magnetization of the pinned layer 231 in one direction (direction of the arrow 234) as shown in FIG. 21, it is possible to provide an antiferromagnetic layer 242 under the pinned layer 231 via a Ru layer 241 of a thickness of 2 nm or less.

This technique is generally referred to as antiferromagnetic material pinning. The magnetization of the outermost surface of the antiferromagnetic layer 242 and the magnetization of the pinned layer 231 are exchange-coupled to each together. Since the magnetization in the antiferromagnetic layer 242 is difficult to be rotated by the external magnetic field, the magnetization of the pinned layer 231 is kept in one direction (direction of the arrow 234). In order to obtain a uni-directional exchange coupling from the antiferromagnetic layer 242, heat treatment in the magnetic field may be used.

The Ru layer 241 is used for exerting the antiferromagnetic exchange-coupling between the outermost surface of the antiferromagnetic layer 242 and the lowermost surface of the pinned layer 231. Also, it used for controlling the exchange coupling strength. However, the pinned layer 231 may be directly placed on the antiferromagnetic layer 242 without using the Ru layer 241. In this case, film structure becomes simple but the exchange coupling becomes difficult to be controlled. A Rh layer or a Cu layer may be used instead of the Ru layer.

As an antiferromagnetic material used for the pinning layer (antiferromagnetic layer) 242, IrMn is preferable since its antiferromagnetic state is stable.

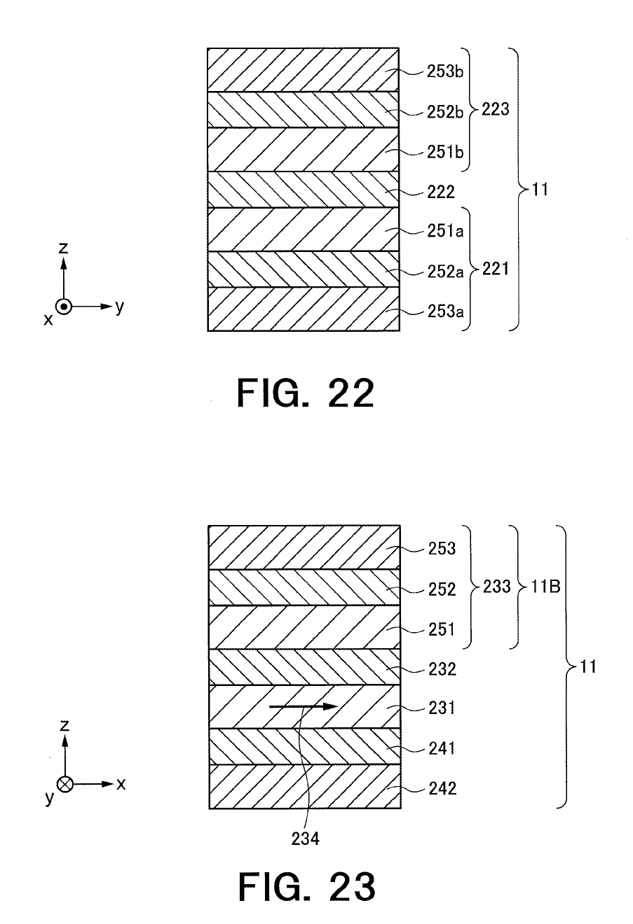

Next, a configuration of the first magnetic layer 221 and the second magnetic layer 223 shown in FIG. 19 is described in detail with reference to FIG. 22. FIG. 22 is a sectional view showing an example of the magnetic field detector 11.

As shown in FIG. 22, the first magnetic layer 221 has a structure in which a soft magnetic high electrical resistance layer 253a and a soft magnetic low electrical resistance layer 251a are stacked via a Ru layer 252a. The soft magnetic high electrical resistance layer 253a and the soft magnetic low electrical resistance layer 251a are antiferromagnetically exchange-coupled together via the Ru layer 252a. The second magnetic layer 223 has a structure in which a soft magnetic low electrical resistance layer 251b and a soft magnetic high electrical resistance layer 253b are stacked via a Ru layer 252b. The soft magnetic low electrical resistance layer 251b and the soft magnetic high electrical resistance layer 253b are antiferromagnetically exchange-coupled together via the Ru layer 252b. The soft magnetic low electrical resistance layers 251a, 251b are faced to the nonmagnetic conductive layer 232.

Next, the free layer 233 shown in FIG. 21 is described with reference to FIG. 23. FIG. 23 is a sectional view showing an example of the magnetic field detector 11. As shown in FIG. 23, the free layer 233 has a structure in which a soft magnetic low electrical resistance layer 251 and a soft magnetic high electrical resistance layer 253 are stacked via a Ru layer 252. The soft magnetic low electrical resistance layer 251 and the soft magnetic high electrical resistance layer 253 are antiferromagnetically exchange-coupled together via the Ru layer 252.

The free layer 233 shown in FIG. 20, similarly to the case shown in FIG. 23, may has a structure in which the soft magnetic low electrical resistance layer 251 and the soft magnetic high electrical resistance layer 253 are stacked via the Ru layer 252, and the soft magnetic low electrical resistance layer 251 and the soft magnetic high electrical resistance layer 253 are antiferromagnetically exchange-coupled together via the Ru layer 252.

For the case of CIP-GMR using the first magnetic layer 221, the second magnetic layer 223, or the free layer 233, conduction electrons are scattered at an interface between the nonmagnetic conductive layer 232 and the soft magnetic layers (first magnetic layer 221, second magnetic layer 223, or free layer 233) lying on and under the nonmagnetic conductive layer 232, and causes a magneto-resistance change. When the thickness of the soft magnetic layer (first magnetic layer 221, second magnetic layer 223, or free layer 233) is increased, thermal fluctuation noise can be reduced, and SNR of the magnetic sensor can be improved.

However, when the thickness of the soft magnetic layer is increased, current also flows inside the soft magnetic layer, and the magneto-resistance ratio is degraded. When the electrical resistance of the soft magnetic layer is increased, the electrons do not easily enter into the soft magnetic material, and then the resistance change is not increased. When the soft magnetic low electrical resistance layers 251a, 251b, 251 and the soft magnetic high electrical resistance layers 253a, 253b, 253 are respectively stacked via the Ru layer 252a, 252b, 252, and the soft magnetic low electrical resistance layers 251a, 251b, 251 are in contact with the nonmagnetic conductive layer 232, the conduction electrons enter the inside of the soft magnetic low electrical resistance layers 251a, 251b, 251 from the interface. Then, the electrons are reflected at the interface with the soft magnetic high electrical resistance layers 253a, 253b, 253, and again return to the nonmagnetic conductive layer 232. As a result, the number of scattering is increased and the resistance change can be increased.

Since the soft magnetic low electrical resistance layers 251a, 251b, 251 are respectively exchange-coupled with the soft magnetic high electrical resistance layers 253a, 253b, 253 via the Ru layer 252, the total magnetic volume is increased and the thermal fluctuation noise can be reduced.

As a material of the soft magnetic low electrical resistance layers 251a, 251b, 251, a FeCo alloy or a Heusler alloy is preferable since the magneto-resistance ratio is high due to high spin polarization. As a material of the soft magnetic high electrical resistance layers 253a, 253b, 253, an amorphous alloy containing CoFeSi, CoFeSiB, CoZrNb, or CoFeB, is preferable. They have an excellent soft magnetic characteristic.

(Magnetic Sensor Production Method)

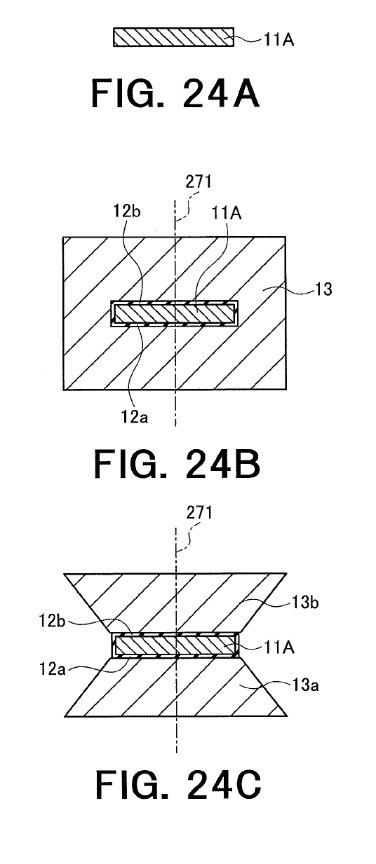

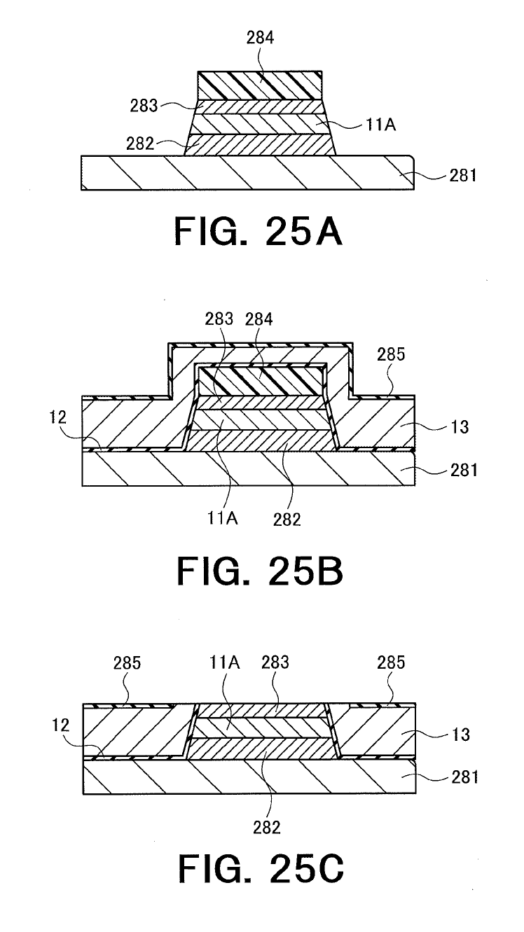

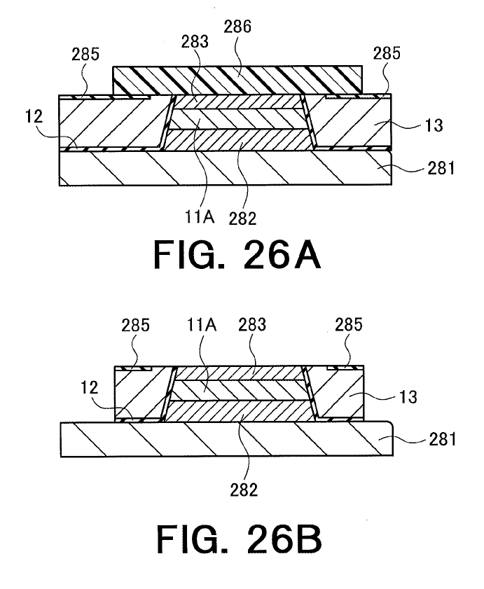

Next, a production method of the magnetic sensor 1 of the first embodiment is described with reference to FIG. 24A to FIG. 26B. FIG. 24A to FIG. 24C are plan views showing the production method. FIG. 25A to FIG. 26B are sectional views cut by a cutting plane line 271 shown in FIGS. 24B, 24C.

First, as shown in FIG. 25A, a base layer 282, magneto-resistive layer 11A, and protective layer 283 are deposited on a substrate 281. The base layer 282 is used for the purpose of controlling crystallinity and/or the magnetic characteristics of the magneto-resistive layer 11A, and/or improving adhesion. The protective layer 283 is deposited for the purpose of suppressing damage to the magneto-resistive layer 11A during a fabrication process or after the formation of the magnetic sensor.

Then, resist layer 284 is applied on the protective layer 283, and then form a resist mask pattern through the exposure and development process. The protective layer 283, the magneto-resistive layer 11A, and the base layer 282 are etched using the mask pattern to form a desired shape. FIG. 24A, FIG. 25A are respectively a plan view and a sectional view showing the layers after being patterned. As shown in FIG. 24A, the magneto-resistive layer 11A is processed into a stripe shape. In general, an etching process through a mask makes the layers a trapezoidal cross-section. FIG. 25A shows this state.

Next, a nonmagnetic insulator 12, a magnetic field concentrator 13, and an insulating protective layer 285 are sequentially deposited without removing the resist pattern. FIG. 25B shows this state. The insulating protective layer 285 is deposited to suppress the process damage during patterning of the magnetic field concentrator. It also prevents unintended electrical short in the fabricated device.

After that, the resist 284 is peeled off, and the nonmagnetic insulator 12, the magnetic field concentrator 13, and the insulating protective layer 285 deposited on the magneto-resistive layer 11A are removed by using a lift-off method. Subsequently, a burr and a residue are removed by polishing, and the surface is flattened. FIG. 24B and FIG. 25C show this state. As described above, since the magneto-resistive layer 11A is patterned into a trapezoidal cross-sectional shape, the nonmagnetic insulator 12, the magnetic field concentrator 13, and the insulating protective layer 285 are deposited on this side walls. After the lift-off process, as shown in FIG. 24B, the nonmagnetic insulator 12 and the magnetic field concentrator 13 appear in the outside portion of the magneto-resistive layer 11A. In FIG. 24B, for the purpose of the ease of the explanation, the insulating protective layer 285 is omitted.

Subsequently, for processing the magnetic field concentrator 13, the resist layer 286 is again applied on the magnetic field concentrator 13 and insulating protective layer 285. The resist layer 286 is exposed and developed, and a resist mask is formed. FIG. 26A shows this state. An etching process is performed through the resist mask 286 to process the magnetic field concentrator 13, and then the resist mask 286 is removed. FIG. 24C and FIG. 26B show this state.

By using the above processes, a distance between the magneto-resistive layer 11A and the magnetic field concentrator 13 can be controlled by a thickness of the nonmagnetic insulator 12. Thus, the distance ranging from a few nm to 50 nm can be easily controlled. This thickness is crucial for the magnetic sensor of the first to second embodiments. In addition, as can be seen from FIG. 24A to FIG. 26B, by providing a base layer and an protective layer to the magneto-resistive layer 11A, a thickness of the magneto-resistive layer 11A can be made to be thinner than that of the magnetic field concentrator 13, and the magneto-resistive layer 11A can be placed at substantially central position in a film thickness direction of the magnetic field concentrator 13. Therefore, a leakage magnetic field from the magnetic charge at the edge of the magneto-resistive layer 11A can be effectively absorbed by the magnetic field concentrator 13, and a demagnetization field can be suppressed.

In a case in which the magneto-resistive layer 11A is processed into a shape shown in FIG. 14 or FIG. 15, the etching mask shown in FIG. 24A is formed to the shape shown in FIG. 14 or FIG. 15. Then, in the process shown in FIG. 26A, 26B, the etching mask is formed so that the magnetic field concentrator 13 is formed only outside the outermost magneto-resistive layer 11A.

In FIG. 24C, the nonmagnetic insulator 12 also remains at the edge of the short axis side of the magneto-resistive layer 11A. When the conducting wire is connected to the magneto-resistive layer 11A for applying electric current, the residual nonmagnetic insulator 12 may be partially removed or avoided. The schematic diagram of FIG. 1 schematically shows a state after such process.

(Noise Level Estimation)

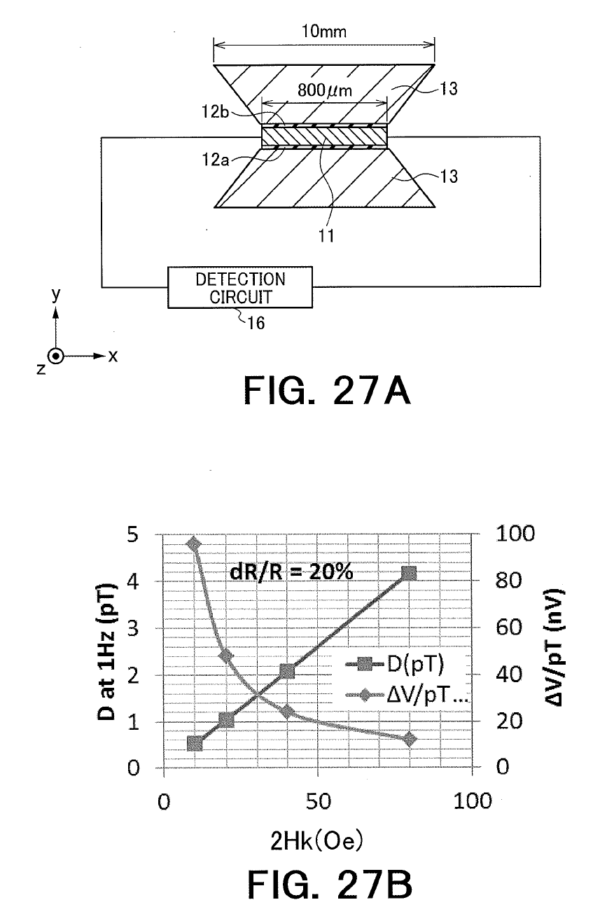

Next, a noise level of the magnetic sensor of the third embodiment has been estimated. FIGS. 27A, 27B shows a result of the estimation. FIG. 27A schematically shows an outline of an assumed magnetic sensor.

The magnetic sensor has a configuration in which the CIP-GMR device 171 shown in FIG. 20 is used as the magneto-resistive layer 11A of the magnetic field detector 11. The CIP-GMR device 171 has a width of 4 μm, a length of 800 μm, and a thickness of 15 nm. A length of each of the magnetic field concentrators 13a, 13b at the magnetic field detector 11 side is 800 μm, a length of an opposite side is 10 mm, a thickness is 600 nm. The magnetic field intensity is assumed to be I magnified by 400 times through the magnetic field concentrators 13a, 13b. It is assumed that a resistance R of the magnetic field detector 11 is 4 kΩ (Johnson noise: about 6 nV), application voltage Vb is 1.2 V (0.36 mW), and a magneto-resistance ratio (dR/R) is 20%. From literature data of AMR, a noise (1/f noise+Johnson noise) at 1 Hz is set to be 50 nV/Hz^{0.5}.

As shown in FIGS. 10 and 11, a saturation field Hk (reciprocal of sensitivity) can be reduced to about a half by making a distance between the magnetic field detector 11 and the magnetic field concentrator 13 50 nm or less (½ or less of a length in the short axis direction of the magnetic field detector 11). Then, it is assumed that 2×Hk is decreased to 80, 40, 20, 10 Oe with the distance between the magnetic field detector 11 and the magnetic field concentrator 13. The ratio of the long axis to the short axis of the soft magnetic material was 10 in the simulation shown in FIGS. 10, 11, while it is 200 in the present estimation. Therefore, further reduction in of Hk is expected here. However, this situation is not considered in the estimation. FIG. 27B shows the change in the output voltage per pT (pico Tesla) and the detection limit D with the 2×Hk. D is the magnetic field strength in which a signal output coincides with the noise level. It is found that detection of the magnetic field less than 1 pT can be expected. This result is due to the fact that low noise and high sensitivity can be achieved by using the magnetic sensor of the first to third embodiments.

### Fourth Embodiment

Next, any of the magnetic sensors of the first to third embodiments can be used for a magnetoencephalograph (MEG) that detects a magnetic field generated by a cranial nerve. This is described as a fourth embodiment.

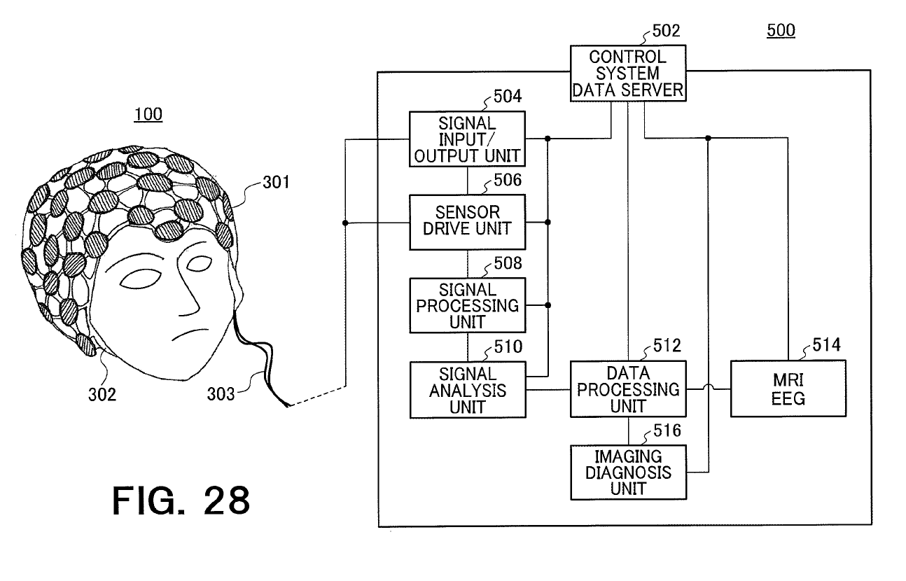

A magnetic sensor apparatus of the fourth embodiment is described with reference to FIG. 28. A magnetic sensor apparatus 100 of the fourth embodiment is a MEG system. Left side figure in FIG. 28 schematically shows an example in which the MEG system 100 is applied to a head of a human body. The MEG system 100 has a plural of sensor units 301 placed on a flexible base body 302.

In each of the sensor units 301, one or a plural of magnetic sensor of any of the first to third embodiments and its modification may be placed. The plural of magnetic sensors may configure a circuit such as differential detection. Other sensors such as potential terminals or acceleration sensors may be installed together. Since the magnetic sensor of the first to third embodiments and the modification thereof is smaller than conventional SQUID magnetic sensor, it is easy to install the multiple sensor units circuit, and other sensors.

The flexible base body 302 is made of, for example, an elastic body such as a silicone resin. Sensor units 301 are arranged in the lines on the flexible base body 302 to be fitted with the head. The base body 302 may be a shape of flexible hat. A net-shaped base body shown in FIG. 28 may be preferable since it has an excellent wearability.

An input/output cord 303 of the sensor units 301 is connected to a sensor drive unit 506 and a signal input/output unit 504 of a diagnosis apparatus 500. The sensor units 301 measure the magnetic field with a power from the sensor drive unit 506 and a control signal from the signal input/output unit 504. A result of the measurement is input to the signal input/output unit 504 in parallel. Then the signal is transmitted from the signal input/output unit 504 to a signal processing unit 508 to be subjected to signal processing such as noise removal, filtering, amplification, and mathematic operation. After that, the signal is subjected to signal analysis such as signal extraction or phase matching in a signal analysis unit 510. Data of the signal analysis is transferred to a data processing unit 512. In the data processing unit 512, image data such as magnetic resonance imaging (MRI) and electroencephalogram (EEG) are incorporated, and data analysis such as neural response analysis and inverse problem analysis is performed. A result of the analysis is transferred to an imaging diagnosis unit 516 for the diagnosis. The above series of operation is controlled by a control system 502. Data such as primary signal data and metadata during data processing is stored in a data server. As shown in FIG. 28, the data server and the control system may be integrated.

The sensor units 301 may be used for a human body chest or for a heart rate test of a fetus.

An entire of the magnetic sensor apparatus including a subject is preferably installed in a shield room to prevent the geomagnetism and magnetic noise. Alternatively, local shielding for a measurement portion of the human body and the sensor units 301 is possible. Shielding may be provided only to the sensor units 301. Effective shielding by the signal processing may be used.

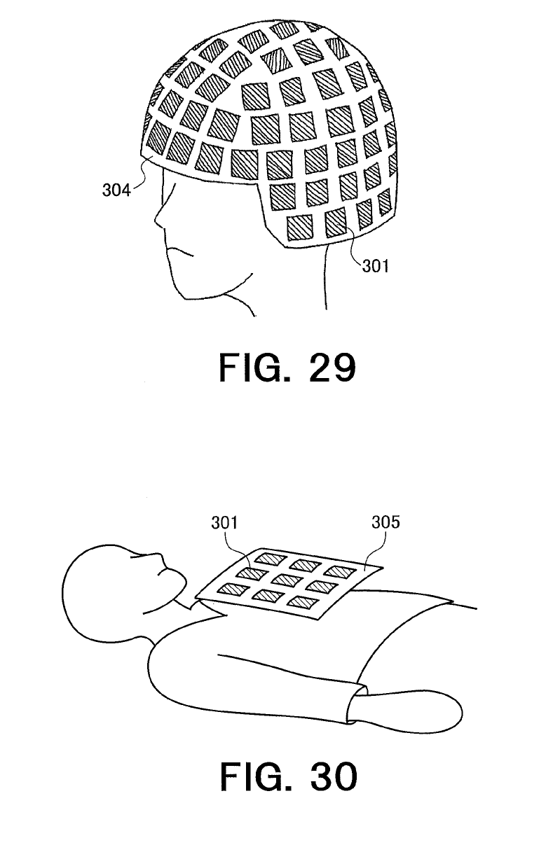

The sensor units 301 may be installed to a fixed base body such as in a conventional magnetoencephalograph or magnetocardiograph. Examples are shown in FIG. 29 and FIG. 30. FIG. 29 shows an example of the magnetoencephalograph, and the sensor units 301 are installed on a helmet-like hard base body 304. FIG. 30 is an example of the magnetocardiograph, and the sensor units 301 are installed on a plate-shaped hard base body 305. In both cases, input/output of a signal from the sensor units 301 and processing of the signal are the same as those in FIG. 28.

(Sensor Unit)

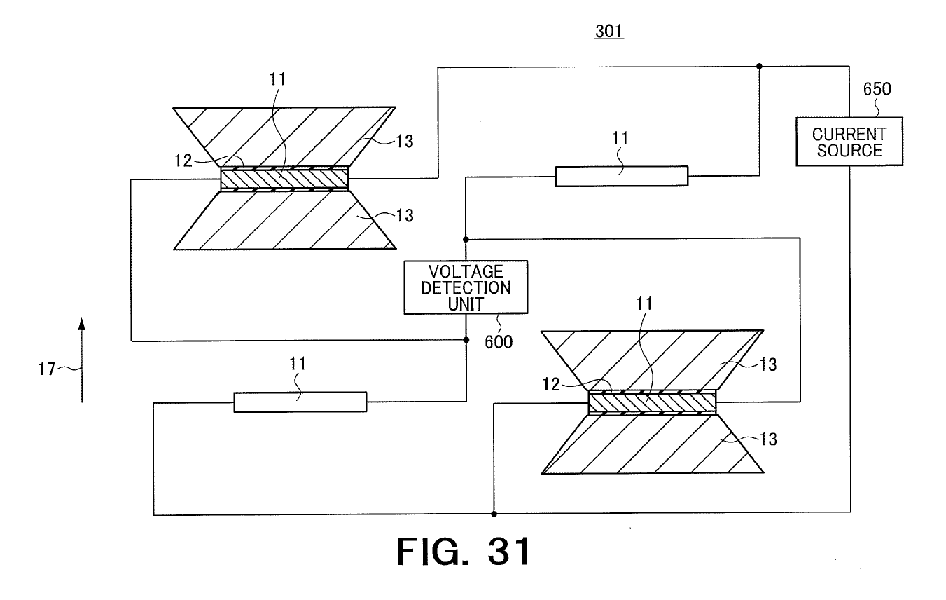

A plural of magnetic sensors may be configured the Wheatstone bridge. An example of the bridge is shown in FIG. 31. Four magnetic field detectors 11 are arranged on a rectangle and are connected as shown in FIG. 31. The direction of a magnetic field to be detected is shown by an arrow 17. The upper left and lower right magnetic field detectors 11 in the FIG. 31 configure the magnetic sensor of any of the first-third embodiments and the modification thereof. Nonmagnetic insulators 12 are provided between the magnetic field detector 11 and the magnetic field concentrator 13.

As described above, the magnetic sensor with the magnetic field concentrators 13 provides a significantly large output signal in comparison with a case of without the magnetic field concentrators. Therefore, in FIG. 31, the upper left and lower right magnetic field detectors 11 show the resistance change with the magnetic field, while the lower left and upper right magnetic field detectors 11 do not show resistance change, that is, become fixed resistances.

When the magnetic field is applied to the configuration in FIG. 31, resistance of the upper left and lower right magnetic field detectors 11 in FIG. 31 are changed, and two times larger output voltage change can be obtained. Since the output voltage is a potential difference between the right side and left side blocks in FIG. 31, noise essentially included in a current source 650 is cancelled. That is, the noise can be suppressed and the output can be increased.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

1. A magnetic sensor comprising:
a magnetic field detector including a magnetic layer in which a length in a first direction is 10 times or more of a length in a second direction perpendicular to the first direction and a length in a third direction perpendicular to the first direction and the second direction is ½ or less of the length in the second direction;
a first magnetic material member that is arranged along the first direction of the magnetic field detector, and in which a length in the third direction is longer than the length in the third direction of the magnetic layer;
a first nonmagnetic insulating layer that is arranged between the magnetic field detector and the first magnetic material member, and in which a length in the second direction is ½ or less of the length in the second direction of the magnetic layer; and
a circuit configured to supply current to the magnetic layer.

2. The sensor according to claim 1, wherein
the magnetic field detector includes a plurality of magnetic layers, and the plurality of magnetic layers is arranged in parallel in the second direction and connected to each other in series.

3. The sensor according to claim 1, wherein
the magnetic field detector comprises at least one current perpendicular to plane magneto-resistive device, the magneto-resistive device includes a first ferromagnetic layer, a second ferromagnetic layer, and a nonmagnetic interlayer provided between the first ferromagnetic layer and the second ferromagnetic layer, the first ferromagnetic layer has a length in the third direction of ½ or less of a length in the second direction, and the first ferromagnetic layer and the second ferromagnetic layer each include an axis of easy magnetization in an in-plane direction.

4. The sensor according to claim 3, wherein
the magnetic field detector includes the plurality of magneto-resistive device connected to each other in series.

5. The sensor according to claim 1, wherein
the magnetic field detector comprises: a nonmagnetic first layer; and a second layer that is arranged on the first layer, and that contains any of a NiFe alloy, a FeCo alloy, and a Heusler alloy, and the second layer has a length in the first direction of 10 times or more of a length in the second direction and has a length in the third direction of ½ or less of the length in the second direction.

6. The sensor according to claim 1, wherein
the magnetic field detector comprises: a first magnetic layer; a second magnetic layer; and a nonmagnetic conductive layer arranged between the first magnetic layer and the second magnetic layer, and the first magnetic layer and the second magnetic layer are antiferromagnetically coupled together via the nonmagnetic conductive layer.

7. The sensor according to claim 6, wherein
the first magnetic layer and the second magnetic layer each comprise: a first soft magnetic layer; a second soft magnetic layer having a higher electrical resistance than an electrical resistance of the first soft magnetic layer; and a nonmagnetic metal layer arranged between the first soft magnetic layer and the second soft magnetic layer, and the first soft magnetic layer and the second soft magnetic layer are antiferromagnetically exchange-coupled together, and the first soft magnetic layer is in contact with the nonmagnetic conductive layer.

8. The sensor according to claim 1, wherein
the magnetic field detector comprises: a pinned layer in which magnetization is pinned in the first direction; a free layer in which an external magnetic field is detected and magnetization is rotated; and a nonmagnetic conductive layer arranged between the pinned layer and the free layer.

9. The sensor according to claim 8, wherein
the magnetic field detector further comprises: an antiferromagnetic layer arranged at an opposite side of the nonmagnetic conductive layer with respect to the pinned layer; and a nonmagnetic metal layer arranged between the pinned layer and the antiferromagnetic layer.

10. The sensor according to claim 8, wherein
the free layer comprises: a first soft magnetic layer; a second soft magnetic layer having a higher resistance than a resistance of the first soft magnetic layer; and a nonmagnetic metal layer arranged between the first soft magnetic layer and the second soft magnetic layer, and the first soft magnetic layer and the second soft magnetic layer are antiferromagnetically exchange-coupled together, and the first soft magnetic layer is in contact with the nonmagnetic conductive layer.

11. The sensor according to claim 6, wherein
any of the free layer, the pinned layer, the first magnetic layer, and the second magnetic layer is a FeCo alloy or a Heusler alloy.

12. The sensor according to claim 7, wherein the first soft magnetic layer is a FeCo alloy or a Heusler alloy.

13. The sensor according to claim 7, wherein
the second soft magnetic layer is amorphous, and contains any of CoFeSi, CoFeSiB, CoZrNb, and CoFeB.

14. The sensor according to claim 6, wherein
the nonmagnetic conductive layer contains Cu.

15. The sensor according to claim 1, further comprising:
a second magnetic material member that is provided at an opposite side of the first magnetic material member with respect to the magnetic field detector, and in which a length in the third direction is longer than the length in the third direction of the magnetic layer; and
a second nonmagnetic insulating layer that is arranged between the magnetic field detector and the second magnetic material member, and in which a length in the second direction is ½ or less of the length in the second direction of the magnetic layer.

16. The sensor according to claim 15, wherein
the first and second nonmagnetic insulating layers each have a length in the second direction of 1 nm or more and 50 nm or less.

17. The sensor according to claim 15, wherein
the first and second magnetic material members each contain any of a NiFe alloy, a FeCo alloy, and a Heusler alloy.

18. A magnetic sensor apparatus comprising:
the magnetic sensor according to claim 1; and
a diagnosis apparatus including a processing analysis circuit for processing and analyzing a magnetic field detection signal from the magnetic sensor, and an imaging circuit for imaging an analysis result of the processing analysis circuit.

19. The apparatus according to claim 18, wherein
the magnetic sensor detects a magnetic field from a brain.

20. The apparatus according to claim 18, wherein
the magnetic sensor detects a magnetic field from a heart.
